(19) United States
(12) Patent Application Publication
(10) Pub. No.: US 2026/0363969 A1
(43) Pub. Date: Nov. 19, 2026

(54) Multi-Phase Oral Care System and Composition for Enamel Protection and Enhanced Dental Hygiene

(71) Applicant: Daniel Cao, Troy, MI (US)

(72) Inventor: Daniel Cao, Troy, MI (US)

(73) Assignee: Daniel Cao, Troy, MI (US)

(21) Appl. No.: 19/206,037

(22) Filed: May 13, 2025

(51) Int. Cl. A61K 8/11; A61Q 11/00

(52) U.S. Cl. CPC A61K 8/11; A61Q 11/00; A61K 2800/56; A61K 2800/884; A61K 2800/92; A61K 2800/95

(57) ABSTRACT

The present invention relates to a multi-phase oral care system designed to enhance the retention and prolonged release of active agents on the enamel surface. The system comprises a first solution (Solution A) containing calcium ions, configured to prepare the enamel by forming adhesive points, and a second solution (Solution B) containing sodium alginate, which, when applied following Solution A, forms a cross-linked hydrogel matrix on the enamel. This matrix encapsulates active agents, such as antimicrobial or breath-freshening compounds, allowing for sustained release and improved oral hygiene. The system can be applied through one of two primary methods: the A+B method, sufficient for extended retention, or the A+B+A method, providing enhanced durability. The hydrogel coating is non-permanent and can be easily removed by brushing. The invention is suitable for various oral care formulations, including mouthwashes, rinses, and gels.

### BACKGROUND OF THE INVENTION

The present invention relates to compositions and methods for oral care, particularly to a multi-phase system designed to enhance the retention of beneficial oral agents through the formation of an alginate-based hydrogel matrix. The invention may be formulated as a mouthwash, oral rinse, oral gel, or other suitable delivery formats, showing a versatile application in various oral hygiene products. The invention is specifically formulated to improve the duration of oral hygiene effects compared to conventional oral care products.

Conventional oral care products—such as mouthwashes and toothpastes—offer various immediate dental benefits, but their effects are typically short-lived. These formulations act during application but are quickly diluted, swallowed, or rinsed away, limiting the duration of their protective activity in the oral cavity.

Xylitol, for example, is a non-cariogenic compound utilized in dental products to support overall oral health, including cavity prevention. However, in standard formulations, its residence time in the mouth is minimal, which reduces the potential long-term impact of its beneficial properties.

There remains a need for an oral hygiene system that extends the retention and effectiveness of beneficial oral compounds over time. A multi-phase application method incorporating an adhesive matrix such as alginate offers a novel approach. In such a system, active agents—such as xylitol—can be adhered to the enamel surface and gradually released over time, potentially throughout the night, thereby prolonging their effects and improving overall dental hygiene.

In addition to xylitol, other active agents can be incorporated into the multi-phase application composition and method. They include, but are not limited to, compounds that support microbial control, plaque inhibition, enamel remineralization, gum health, and breath freshening. This results in formulations tailored to specific oral care needs.

### SUMMARY OF THE INVENTION

The present invention provides a multi-phase oral care system and composition designed to enhance the retention and sustained release of beneficial oral agents on tooth surfaces. As depicted in FIG. 1, the system comprises at least two distinct formulations—referred to as Solution A and Solution B—that are sequentially applied to maximize the effectiveness of active ingredients.

Solution A is a calcium-containing pre-treatment that prepares the enamel surface by creating adhesive points conducive to binding. These points facilitate the subsequent attachment of Solution B, thereby prolonging the retention of active ingredients.

Solution B is the primary treatment composition, containing an alginate-based polymer in a viscous, free-flowing form. Upon contact with the calcium ions from Solution A, the alginate undergoes ionic cross-linking, forming a hydrogel matrix that adheres to the prepared enamel surface. The matrix acts as a sticky layer that remains on the teeth for an extended period, retaining and gradually releasing the encapsulated functional agents—such as xylitol for microbial control. This controlled, slow-release mechanism ensures that active ingredients remain effective over a prolonged duration.

An important aspect of this invention is the practical removability of the alginate-calcium hydrogel matrix. Despite its adhesive properties, the matrix is easily dislodged from the tooth surface with standard brushing, ensuring that no residue remains after routine oral hygiene practices.

In addition to xylitol, this system may incorporate a wide range of functional agents including—but not limited to—compounds that support gum health, enamel remineralization, microbial control, breath freshening, and plaque inhibition. This adaptability allows the formulation to be customized according to specific oral care needs.

The multi-phase application method allows for two primary sequences:
1. A+B: Solution A is applied to prepare the enamel, followed by Solution B, whose alginate polymer cross-links and adheres to the treated surface, retaining active agents while gradually releasing them.
2. A+B+A: After applying Solution A and then Solution B, a second application of Solution A can be performed to further strengthen the hydrogel matrix and enhance the retention of the active agents, thereby prolonging the effective duration of the system.

The key innovation of this system lies in the use of a calcium-alginate strengthened hydrogel matrix formed through sequential applications of Solutions A and B, which forms upon application to the enamel, securely holding the active ingredients while allowing controlled, slow release over an extended period. This configuration significantly extends the duration of action, allowing beneficial agents to remain effective on the teeth for prolonged periods—potentially overnight—thereby providing sustained oral health benefits beyond those of conventional single-phase oral care products.

Due to its modular nature, the system can be adapted for various formulations, targeting specific dental health needs by incorporating different active ingredients in Solution B. This flexibility makes it suitable for use as a standalone oral hygiene product or as an adjunct to traditional dental care routines.

The invention can optionally be used in a variety of oral care formats, including but not limited to mouthwashes, gels, oral rinses, or other dental hygiene products. The formulation's versatility allows it to be incorporated into diverse oral care routines to significantly improve agent retention and efficacy compared to current state-of-the-art products.

### Safety Considerations

Alginate is widely recognized for its biocompatibility and safety. It is commonly used as a thickener and stabilizer in the food industry and in biomedical applications such as wound dressings and drug delivery systems. In the context of oral care, alginate is considered safe for human use, and incidental ingestion in small amounts is generally regarded as harmless.

### BRIEF DESCRIPTION OF DRAWINGS

The accompanying drawings illustrate various embodiments and application methods of the present invention, in which:

FIG. 1: A schematic diagram illustrating the multi-phase oral care system, including the sequential use of Solution A, Solution B and Solution A (optional re-application). This diagram represents the overall concept and workflow of the invention, highlighting the application sequence and composition for each solution.

FIG. 2: An illustration of Solution A being applied to the enamel surface, creating adhesive points through the deposition of calcium ions (Ca²⁺) (illustrated by black dots). The figure shows how the ions interact with the enamel to prepare it for subsequent hydrogel formation when Solution B is applied.

FIG. 3: A depiction of Solution B being applied after Solution A, forming a hydrogel matrix on the enamel surface. The diagram shows how sodium alginate in Solution B (illustrated by lines) interacts with pre-deposited calcium ions to create a cross-linked hydrogel that adheres to the tooth surface.

FIG. 4: A detailed view of the hydrogel matrix structure, illustrating how active agents (e.g., antimicrobial agents, flavoring agents) are encapsulated and gradually released. The figure highlights the network of cross-links that stabilize the hydrogel, ensuring prolonged retention and controlled release of the encapsulated agents.

FIG. 5: The A+B+A application sequence, illustrating how the second application of Solution A after Solution B further strengthens the hydrogel matrix. The figure demonstrates the increased density of cross-links, especially upon the surface of the hydrogel matrix. This additional cross-linking step further enhances the retention of active agents.

FIG. 6: A demonstration of the removal process, showing how simple brushing effectively removes the hydrogel layer from the enamel surface. The figure highlights the non-permanent adhesion of the cross-linked hydrogel, emphasizing that the coating can be easily eliminated through normal oral hygiene practices.

FIG. 7: A theoretical comparison chart representing the expected retention time and controlled release of active agents in the multi-phase oral care system versus conventional single-phase oral care formulations. The graph clearly shows both A+B and A+B+A methods significantly outperform traditional products that lack prolonged retention. Additionally, this chart suggests that the A+B+A method provides further improved retention compared to the A+B method.

### DETAILED DESCRIPTION OF THE INVENTION

### Detailed Description of Solution A

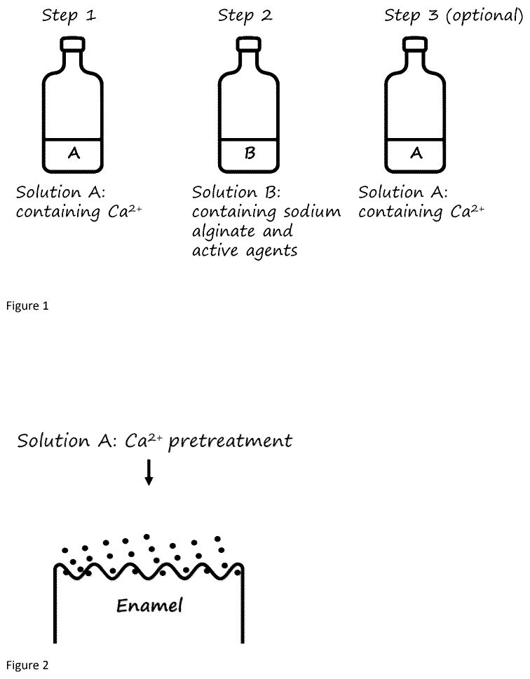

Solution A functions as the calcium-containing pre-treatment in the multi-phase oral care system. As depicted in FIG. 2, the primary purpose of Solution A is to prepare the enamel surface by creating adhesive points through the application of Ca²⁺ ions. These points facilitate the subsequent attachment of Solution B, thereby prolonging the retention of active ingredients and forming a strengthened hydrogel matrix.

### Primary Functional Component: Calcium Ions (Ca²⁺)

The essential component of Solution A is Ca²⁺ ions, which are responsible for cross-linking with alginate in Solution B to form a robust hydrogel matrix.

Solution A comprises calcium ions derived from calcium chloride (CaCl₂) or calcium acetate (Ca(C₂H₃O₂)₂). These calcium sources are chosen for their high solubility and dissociation efficiency, which ensures maximum availability of Ca²⁺ ions for cross-linking with the alginate matrix.

Alternative calcium sources may also be employed, although they are less preferred due to lower dissociation efficiency, which may result in reduced hydrogel formation. These alternative calcium sources include but are not limited to calcium lactate (Ca(C₃H₅O₃)₂) and calcium gluconate (Ca(C₆H₁₁O₇)₂).

### Calcium Ion Concentration

The concentration of calcium ions in Solution A ranges from 0.5 mM to 5.0 mM. This range is effective for forming a cross-linked alginate hydrogel.

Even at low concentrations, Ca²⁺ ions are capable of binding to enamel proteins and hydroxyapatite, resulting in a surface-enrichment layer. This interaction with the enamel surface can lead to localized calcium concentration, even when the overall Ca²⁺ concentration in Solution A is low.

Due to this enrichment effect, even highly diluted Ca²⁺ solutions (as low as 1 mM, 0.5 mM, or even lower) may still effectively facilitate hydrogel formation. This property makes even diluted Ca²⁺ solutions effective when applied directly to tooth surfaces, minimizing taste interference and reducing potential irritation.

### Optional Non-Essential Components

Solution A may optionally comprise one or more non-essential ingredients to enhance user experience. The presence of these ingredients does not significantly alter the primary function of delivering calcium ions. The choice and concentration of these optional components may vary based on formulation preferences and regulatory requirements. Their inclusion does not affect the primary function of calcium delivery and hydrogel formation.

Preservatives may be included to maintain microbial stability. Examples of suitable preservatives include but are not limited to sodium benzoate, potassium sorbate, ethylparaben, methylparaben, and chlorhexidine gluconate.

Sweeteners may optionally be included to enhance palatability. Suitable sweeteners include but are not limited to xylitol, sorbitol, sucralose, stevia extract, and erythritol.

Viscosity modifiers can optionally be used to improve texture and application properties. Suitable viscosity modifiers include but are not limited to glycerin, propylene glycol, hydroxyethylcellulose, xanthan gum, and polyethylene glycol (PEG).

Colorants may be added to enhance visual appeal. Suitable colorants include but are not limited to FD&C Blue No. 1, FD&C Green No. 3, FD&C Red No. 40, and beet juice powder.

Cooling agents can optionally be used to provide a refreshing sensation. Suitable cooling agents include but are not limited to menthol, eucalyptus oil, menthyl lactate, WS-23 (coolant), and peppermint oil.

Additional additives may be included to enhance stability or sensory appeal. These may include but are not limited to essential oils (such as spearmint or cinnamon), polysorbate 20, and fragrance compounds.

### Detailed Description of Solution B

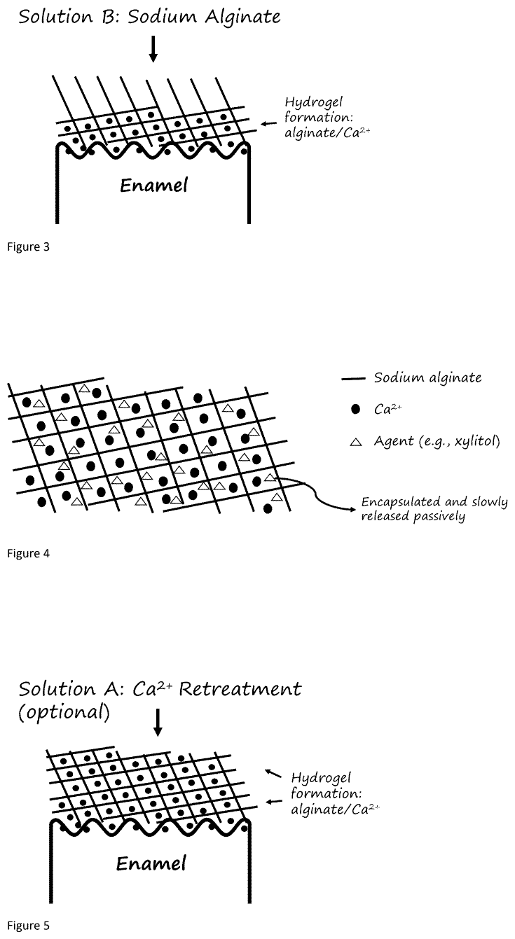

Solution B functions as the second phase in the multi-phase oral care system. Its primary purpose is to deliver active agents upon forming a sustained-release hydrogel. The hydrogel formation occurs only when Solution B is applied following the treatment with Solution A, which contains calcium ions. As depicted in FIG. 3, The combination of the two solutions results in a cross-linked alginate hydrogel that adheres to the enamel surface, allowing for prolonged retention and controlled release of active ingredients.

### Primary Functional Component: Sodium Alginate (Alginate Polymer)

The essential component of Solution B is sodium alginate, which serves as the polymer that forms the hydrogel. Sodium alginate, when exposed to calcium ions from Solution A, undergoes a cross-linking reaction, forming a strengthened hydrogel that adheres to the enamel surface. This hydrogel provides a slow-release platform for active agents, which are free-flowing within Solution B until being encapsulated during the gelation process.

Solution B primarily comprises sodium alginate in concentrations ranging from 0.5% to 2% (w/v). This concentration range ensures adequate viscosity and efficient hydrogel formation when combined with calcium ions from Solution A. Sodium alginate is chosen for its biocompatibility, ease of gelation with calcium ions, and ability to form a stable hydrogel under oral conditions. The use of sodium alginate is advantageous due to its edibility and safety for oral applications, making it suitable for use in oral care products such as mouthwash or oral rinse.

### Active Agents: Xylitol and Other Functional Components

Solution B is formulated to deliver active agents that can provide antimicrobial, protective, or hygiene-enhancing effects. One notable active agent is xylitol, known for its ability to inhibit microbial growth and reduce the risk of dental caries.

### Varying Xylitol Concentration

The concentration of xylitol in commercially available mouthwash products typically ranges from 5% to 25% or even higher, depending on the intended level of antimicrobial action and taste enhancement. In the context of this invention, lower concentrations of xylitol (0.5% to 5% or lower) may also be effective due to the prolonged retention provided by the hydrogel matrix. The slow release of xylitol from the hydrogel allows even reduced xylitol concentrations to maintain microbial control over extended periods. Therefore, both high and low xylitol concentrations are considered suitable and are reflected in the formulation examples.

Both with and without xylitol, Solution B may optionally comprise of other active agents, depending on the desired functional profile. These may include other compounds for microbial control (including but not limited to chlorhexidine), enamel remineralization (including but not limited to fluoride compounds), gum health (including but not limited to aloe vera extract), or breath freshening (including but not limited to essential oils). The inclusion of multiple active agents should take compatibility and stability into account within the alginate polymer system.

### Optional Non-Essential Components

Solution B may optionally comprise one or more of the following non-essential ingredients to enhance sensory appeal, stability, or functional performance. The presence of these ingredients does not significantly alter the primary function of hydrogel formation and active agent delivery.

Preservatives may be included to ensure microbial stability. Suitable preservatives include but are not limited to sodium benzoate, potassium sorbate, ethylparaben, methylparaben, and chlorhexidine gluconate.

Sweeteners may optionally be included to improve palatability. Suitable sweeteners include but are not limited to xylitol, sorbitol, sucralose, stevia extract, and erythritol.

Viscosity modifiers may be added to adjust the texture and application properties. Suitable viscosity modifiers include but are not limited to glycerin, propylene glycol, hydroxyethylcellulose, xanthan gum, and polyethylene glycol (PEG).

Colorants may be added to enhance visual appeal. Suitable colorants include but are not limited to FD&C Blue No. 1, FD&C Green No. 3, FD&C Red No. 40, and beet juice powder.

Cooling agents can optionally be used to provide a refreshing sensation. Suitable cooling agents include but are not limited to menthol, eucalyptus oil, menthyl lactate, WS-23 (coolant), and peppermint oil.

Additional additives may include stabilizing or flavor-enhancing compounds, including but not limited to essential oils (like spearmint or cinnamon), polysorbate 20, and fragrance compounds.

### Cross-Linking Process

The cross-linking process is not inherent to Solution B alone but occurs upon application following Solution A. When Solution B, containing sodium alginate, contacts the enamel surface pre-treated with calcium ions from Solution A, ionic cross-linking occurs. This results in the formation of a thin, adherent hydrogel layer that traps and gradually releases the active agents.

The hydrogel matrix formed through the interaction of sodium alginate and calcium ions is localized to the enamel surface. This adhesion is crucial for long-lasting protection and localized therapeutic effects.

As depicted in FIG. 4, Solution B is specifically formulated to interact with calcium ions delivered by Solution A, forming a cross-linked alginate hydrogel on the enamel surface. This hydrogel acts as a slow-release platform for active agents, allowing for prolonged therapeutic effects. The use of sodium alginate ensures biocompatibility and stability, while optional components enhance flavor, texture, and antimicrobial efficacy. Active agents within Solution B remain free-flowing until gelation occurs upon application following Solution A.

The cross-linking process in the multi-phase oral care system is a critical mechanism that enables the formation of a sustained-release hydrogel matrix on the enamel surface to achieve the preferred local effect. This process does not occur within Solution B itself but rather upon the sequential application of Solution A followed by Solution B. The cross-linking reaction specifically takes place when sodium alginate from Solution B encounters calcium ions (Ca²⁺) previously deposited on the enamel surface by Solution A.

Additionally, as depicted in FIG. 5, the system may involve an A+B+A type of application, where reapplication of Solution A after Solution B further cross-links the surface of the adhered alginate polymer layer. This additional step can enhance the durability and retention of the hydrogel on the enamel surface.

### Process Overview

### A+B Application Process:

The cross-linking process is initiated when Solution A, containing Ca²⁺ ions, is first applied to the enamel surface. These calcium ions are deposited onto the enamel, forming adhesive points that serve as cross-linking sites. Following this application, Solution B, which contains sodium alginate and active agents, is introduced. As the alginate polymer in Solution B comes into contact with the calcium ions from Solution A, ionic cross-linking occurs. This interaction results in the formation of a cross-linked alginate hydrogel, which adheres to the enamel surface.

The hydrogel formation is localized to the enamel surface. This selective adhesion ensures that the active agents are retained specifically at the site of application, providing targeted therapeutic effects.

### A+B+A Application Process:

In addition to the standard A+B application, the multi-phase system may also utilize an A+B+A application method. This approach involves a reapplication of Solution A after the initial application of Solution B.

After the cross-linked hydrogel has formed on the enamel surface, the reapplication of Solution A introduces additional calcium ions, which interact with the exposed functional groups of the alginate polymer layer. This second cross-linking phase reinforces the outer surface of the hydrogel, resulting in a more robust and durable matrix.

### Mechanism of Hydrogel Formation:

The primary mechanism of hydrogel formation is driven by the ionic interaction between Ca²⁺ ions and sodium alginate. Calcium ions, previously deposited on the enamel by Solution A, act as cross-linking agents that interact with the carboxyl groups of the alginate polymer chains. This interaction forms ionic bridges, resulting in a gel-like network that adheres to the tooth surface.

The gelation reaction is both rapid and localized, occurring precisely where the calcium ions are present. The structure of the hydrogel matrix features calcium-alginate cross-links, creating a three-dimensional network that traps the active agents present within Solution B.

The hydrogel formation is facilitated by the high solubility and dissociation efficiency of the preferred calcium salts (such as calcium chloride and calcium acetate). These salts provide readily available Ca²⁺ ions, which are essential for efficient cross-linking.

### Role of Active Agents:

During the formation of the cross-linked hydrogel, the active agents present in Solution B become entrapped within the alginate matrix. Prior to the gelation process, these agents remain free-flowing within Solution B. Once the hydrogel forms, the active agents are mechanically, and in certain conditions, chemically integrated into the matrix, allowing for passive and gradual release as the hydrogel slowly degrades or as ions are exchanged with the oral environment.

This controlled release mechanism is particularly beneficial for antimicrobial agents like xylitol, as it allows for prolonged microbial control despite the use of lower concentrations. The gel matrix serves as a reservoir, releasing the active components over an extended period, which significantly enhances the duration of oral protection compared to conventional mouthwash formulations.

Separating the cross-linking process from the formulation of Solution B offers a key advantage. Solution B remains chemically and physically stable and fluid prior to application, preventing premature gelation or consistency issues.

### Removal of the Hydrogel Coating:

The hydrogel coating formed through the cross-linking process is specifically designed to be non-permanent and easily removable through standard oral hygiene practices. The calcium-alginate hydrogel matrix adheres to the enamel surface primarily through ionic interactions, which, while strong enough to retain active agents, do not create permanent bonding.

Even when the A+B+A method is used, forming a more densely cross-linked hydrogel, the resulting coating remains brushable and can be effectively removed with mechanical brushing or standard oral cleaning routines. The slightly increased resistance of the A+B+A hydrogel may require more thorough brushing compared to the A+B hydrogel, but both are safely and completely removable without damaging the enamel surface.

This feature is illustrated in FIG. 6, which demonstrates the removal process of the hydrogel coating from the enamel surface using a toothbrush. The figure highlights the biocompatibility of the hydrogel and its non-permanent adhesion, emphasizing that the coating does not pose risks of long-term accumulation.

The ability to easily remove the hydrogel after its intended period of use ensures that the oral care system is both effective and consumer-friendly, preventing issues related to residual buildup or excessive adhesion.

### Agent Release Behavior:

The agent release behavior of the multi-phase oral care system is significantly improved compared to conventional mouthwash formulations. Current commercial mouthwashes generally exhibit no retention of active agents after application, with their effects being immediate and transient. In contrast, the present invention, utilizing the A+B and A+B+A methods, demonstrates substantially superior retention, enabling prolonged therapeutic effects that significantly exceed the current state of the art.

### Comparison Between A+B and A+B+A Methods

The A+B method involves the sequential application of Solution A followed by Solution B, forming a cross-linked alginate hydrogel on the enamel surface. This hydrogel structure effectively retains active agents for an extended period, well beyond the immediate effect seen in conventional mouthwashes. The encapsulated agents, including but not limited to xylitol, are gradually released as the hydrogel undergoes degradation or ion exchange within the oral cavity.

The A+B method alone can be sufficient for prolonged retention, including potential overnight protection. The hydrogel formed in this process demonstrates sufficient adhesion and stability to maintain the release of active agents for a notably extended duration, significantly outperforming standard mouthwash formulations that lose efficacy almost immediately.

The A+B+A method includes an additional reapplication of Solution A after the initial application of Solution B. This second application of calcium ions further cross-links the surface of the already formed hydrogel, creating a more densely cross-linked outer layer. While this additional step improves durability and retention, it may also make the hydrogel slightly more resilient to removal, requiring a more thorough brushing.

Both methods represent a substantial improvement over existing mouthwash products, which typically provide no lasting retention after use. Even the A+B method alone already demonstrates significant advancement in maintaining active agent presence on the enamel surface. The A+B+A method offers additional reinforcement for situations where maximum durability and retention are desired.

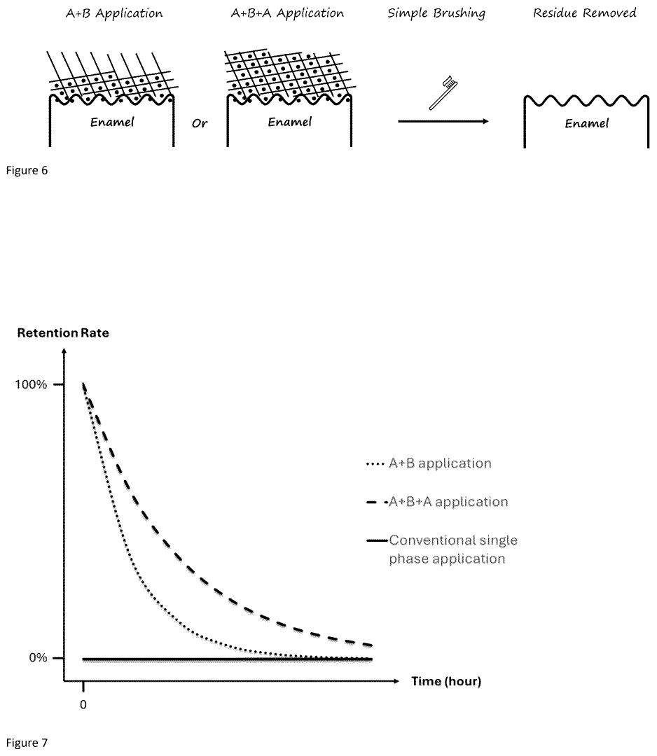

This difference is illustrated in FIG. 7, which shows a comparative analysis of agent retention between the A+B and A+B+A application methods. As depicted, the A+B+A mouthwash formulation demonstrates enhanced retention of encapsulated agents due to the increased level of alginate cross-linking achieved with the additional calcium application. However, both methods significantly outperform conventional mouthwashes, which typically retain no active agents beyond initial application.

### Possibility of Extended Sequences (A+B+A+B or A+B+A+B+A):

It is theoretically possible for users to perform extended application sequences, such as A+B+A+B or A+B+A+B+A. These sequences involve alternating applications of Solutions A and B to increase the density of the cross-linked hydrogel. While this approach could theoretically lead to even longer retention and greater durability, it is not preferred due to the complexity of application and potential difficulty in removal.

However, since the proposed method is consumer-applied, some users may choose to adopt these extended sequences. In such cases, the resulting hydrogel matrix would be increasingly cross-linked, potentially further enhancing the slow-release properties of the encapsulated agents. Nonetheless, the core invention focuses on the A+B and A+B+A methods as the most practical and efficient solutions.

### Mechanism of Agent Retention:

In both methods, the cross-linked hydrogel matrix serves as a physical barrier, gradually releasing the encapsulated agents through diffusion and controlled degradation. The A+B+A method exhibits a slower release rate due to the additional cross-linking on the outer surface of the hydrogel. This enhanced cross-linking results in greater resistance to mechanical forces and saliva dilution, making it more suitable for prolonged exposure.

However, the A+B method alone is sufficient for substantial retention, significantly outperforming typical mouthwashes that lose efficacy almost immediately. The hydrogel formed in the A+B method is robust enough to withstand normal oral activities, maintaining the release of agents, including but not limited to xylitol for a notably extended duration.

### Advantages of Both Methods:

Advantages of A+B Method. It provides long-lasting retention, significantly beyond the immediate effects seen with standard formulations. It involves only two steps, making it more user-friendly for regular oral care routines. The cross-linked hydrogel formed in the A+B method is adequate for long-lasting protection, maintaining agent presence significantly better than conventional products. The hydrogel layer formed using this method is less resilient, allowing for easier removal through brushing.

Advantages of A+B+A Method. The additional cross-linking step results in longer retention of active agents on the enamel surface, particularly beneficial for extended protection. The reinforced hydrogel layer is more resistant to saliva dilution and mechanical forces. The gradual release of active agents, including but not limited to xylitol, provides sustained antimicrobial effects and enhanced enamel protection. It is suitable when long-lasting oral care is specifically desired, such as in cases requiring greater resistance to disruption. The enhanced cross-linking may require more thorough brushing to completely remove the hydrogel layer.

### Performance Characteristics:

Both methods offer localized adhesion to the enamel surface, allowing for slow and controlled release of active agents. The A+B method features ease of use and sufficient retention, while the A+B+A method is favored for maximum durability and retention. The choice between the two methods can be based on user preference and the desired duration of protection.

While extended sequences such as A+B+A+B or A+B+A+B+A are possible, they are not preferred due to their complexity and the potential for excessive hydrogel buildup, which may require more rigorous removal. Nonetheless, acknowledging their feasibility is important, as some consumers may adopt such patterns to achieve extended agent retention.

### Application Method:

The multi-phase oral care system allows for two primary application methods, tailored to user preference and desired duration of protection.
1. A+B Method:
   Step 1: Apply Solution A to the enamel surface, allowing the calcium ions to form adhesive points.
   Step 2: Follow with Solution B, which reacts with the pre-deposited calcium ions, forming a cross-linked hydrogel. This method is efficient and suitable for most oral care needs, providing significant retention of active agents compared to conventional oral care products.
2. A+B+A Method:
   Step 1: Apply Solution A to the enamel surface.
   Step 2: Apply Solution B to initiate cross-linking.
   Step 3: Reapply Solution A to enhance surface cross-linking and durability. This method is ideal for scenarios requiring maximum retention of active agents.

Both methods significantly outperform conventional oral products including but not limited to mouthwashes, which typically offer no retention after use. While the A+B method provides adequate protection for extended periods, the A+B+A method offers enhanced durability. Users may select either method based on convenience and intended application duration.

### EXAMPLES

### Hypothetical Examples of Solution A

In one example, Solution A consists of calcium chloride (CaCl₂) in a concentration range of 0.5 mM to 5.0 mM and purified water. It may optionally include a preservative, such as sodium benzoate in a concentration range of 0.05% to 0.1%, and a flavoring agent, such as peppermint oil in a concentration range of 0.01% to 0.05%. This formulation features its clear and primary function of calcium ion delivery.

According to another example, Solution A comprises of calcium chloride (0.5 mM to 5.0 mM), sorbitol (1% to 5%), natural mint flavor (0.01% to 0.05%), green food dye (trace amount), and purified water. It is formulated to balance calcium delivery with user-friendly sensory characteristics.

In a further example, Solution A contains calcium acetate (0.5 mM to 5.0 mM), menthol (0.01% to 0.1%), eucalyptus extract (0.05% to 0.2%), xylitol (0.5% to 2%), blue dye (trace amount), and purified water. It is designed to provide a cooling and refreshing experience.

In an additional example, Solution A includes calcium chloride (0.5 mM to 5.0 mM), glycerin (1% to 5%), vanilla flavor (0.01% to 0.05%), and purified water. It is formulated to offer a smoother texture, and a mild taste.

### Hypothetical Examples of Solution B

In one example, Solution B consists of sodium alginate at a concentration of 1% (w/v) and xylitol at a concentration of 5%. The solution may include purified water as the solvent. Optional additives may include a sweetener such as sorbitol and a preservative such as sodium benzoate. This formulation is designed for basic antimicrobial protection.

In another example, Solution B comprises sodium alginate at 1.5% (w/v) and xylitol at a concentration of 25% to maximize antimicrobial action. Optional components may include peppermint oil for flavor and glycerin to improve viscosity. This formulation is intended for enhanced microbial control.

In a further example, Solution B includes sodium alginate at 1% (w/v), chlorhexidine at 0.12%, menthol at 0.05%, and eucalyptus oil at 0.02%. Optional ingredients may include sorbitol for sweetness and a trace amount of blue dye for visual appeal. This formulation is designed to provide prolonged freshness and antimicrobial protection (gum health).

In an additional example, Solution B contains sodium alginate at 0.75% (w/v), xylitol at 5% for microbial control, aloe vera extract at 5% for gum health, and menthol at 0.05% for a cooling effect. The solution may optionally contain a preservative, such as potassium sorbate, and a natural mint flavor for improved palatability. This formulation is aimed at providing comprehensive oral hygiene and lasting freshness.

### Hypothetical Example: Xylitol Retention on Model Enamel Substrate

The following hypothetical example illustrates a potential method for assessing xylitol retention using a model enamel substrate. This example is intended to demonstrate the theoretical retention capabilities of the multi-phase oral care system, rather than presenting experimentally verified results.

### 1. Application of Solution A:

In a hypothetical scenario, a model enamel substrate made of hydroxyapatite is utilized to simulate the surface properties of natural tooth enamel. The model enamel substrate is first treated with Solution A, containing calcium ions (Ca²⁺) at a concentration of 5 mM, through immersion for 30 seconds. The calcium ions are expected to be deposited on the enamel surface, forming adhesive points that facilitate the subsequent cross-linking reaction.

### 2. Application of Solution B:

Following the application of Solution A, the substrate is treated with Solution B, containing sodium alginate (1% w/v) and xylitol (25% w/v), through immersion for 30 seconds. The high xylitol concentration is chosen in this hypothetical example to demonstrate the maximum potential retention effect. Upon contact with the pre-treated surface, the sodium alginate undergoes ionic cross-linking with the deposited calcium ions, forming a cross-linked hydrogel layer that adheres to the enamel surface.

### 3. Potential Re-application of Solution A:

In the alternative A+B+A method, the substrate is further treated with Solution A, containing calcium ions (Ca²⁺) at a concentration of 5 mM, through immersion for 30 seconds. The calcium ions are expected to further enhance the crosslinking of alginate hydrogel surface.

### 4. Xylitol Retention Measurement:

The treated substrate through A+B or A+B+A methods is then immersed in a saliva-mimicking solution to simulate oral conditions. Xylitol release is monitored over a 12-hour period to assess the retention performance. At 1, 2, 4, 6, 8, and 12 hours post-immersion, the xylitol concentration in the saliva-mimicking solution is determined using a Xylitol Assay Kit (K-SORB from Megazyme). The Amount Released for xylitol is calculated. In a control study, the treated substrate is immersed under sonication for 15 minutes to fully release the retained xylitol. The Entire Amount Retained is determined, and the percentage retention at each time point is calculated using the formula:

Percentage Retention=(Entire Amount Retained−Amount Released)/Entire Amount Retained

### 5. Expected Results:

Based on the known cross-linking properties of calcium-alginate hydrogels, it is theoretically expected that xylitol will be retained within the hydrogel matrix, providing a gradual and sustained release compared to conventional oral care formulations. It is anticipated that the A+B method will demonstrate adequate retention over time, while the A+B+A method will show enhanced durability and longer retention due to the additional cross-linking step. In comparison to standard mouthwash formulations, which typically exhibit immediate agent loss, the proposed system is expected to significantly extend the presence of xylitol on the enamel surface.

### 6. Rationale:

This hypothetical example demonstrates the expected practical functionality of the multi-phase oral care system based on the scientifically established mechanism of calcium-alginate cross-linking. The described method highlights the anticipated advantage of sustained xylitol release, contributing to improved oral hygiene and microbial control.

1. A multi-phase oral care system comprising:
Solution A, formulated to deliver calcium ions (Ca²⁺) to an enamel surface, thereby forming adhesive points;
Solution B, formulated to contain sodium alginate and one or more active agents, in which Solution B, upon application following Solution A, forms a cross-linked hydrogel on the enamel surface, encapsulating the active agents for prolonged retention and release.

2. The multi-phase oral care system of claim 1, in which Solution A comprises:
A calcium salt selected from calcium chloride or calcium acetate, in which the calcium ions are present at a concentration ranging from 0.1 mM to 5.0 mM, preferably from 0.5 mM to 5.0 mM;
An aqueous base suitable for oral application;
Optional non-essential additives, including preservatives, sweeteners, viscosity modifiers, colorants, and cooling agents.

3. The multi-phase oral care system of claim 1, in which Solution B comprises:
Sodium alginate as the primary polymer at a concentration of 0.5% to 2% (w/v);
One or more active agents selected from xylitol, antimicrobial compounds, fluoride compounds, aloe vera extract, or essential oils;
An aqueous medium suitable for forming a hydrogel when applied after Solution A;
Optional non-essential additives, including preservatives, sweeteners, viscosity modifiers, colorants, and cooling agents.

4. A method of enhancing oral hygiene through the multi-phase oral care system of Claim 1, comprising the steps of:
Applying Solution A to the enamel surface, allowing calcium ions to form adhesive points;
Applying Solution B after Solution A to initiate cross-linking, forming a hydrogel that traps the active agents.

5. The method of claim 4, further comprising:
Reapplying Solution A after the formation of the hydrogel to further cross-link the surface, increasing the durability and retention of the hydrogel layer.

6. The multi-phase oral care system of claim 1, in which the formulation may be presented in various formats, including mouthwash, oral rinse, gel, spray, or other suitable delivery formats.

7. The multi-phase oral care system of claim 1, in which the cross-linked hydrogel formed through the A+B and A+B+A methods provides prolonged retention of active agents relative to non-crosslinked oral care formulations.

8. The method of claim 4, in which the application sequence may include additional cycles such as A+B+A+B or A+B+A+B+A, thereby enhancing the retention and durability of the hydrogel.

9. The multi-phase oral care system of claim 1, in which the cross-linked hydrogel matrix formed on the enamel surface provides:
Sustained retention and prolonged release of active agents over an extended duration;
Localized adherence to the enamel, resisting displacement by saliva or oral movements.

10. The multi-phase oral care system of claim 3, in which the active agents are gradually released through diffusion from the cross-linked hydrogel matrix, maintaining antimicrobial effectiveness and enamel protection over an extended period.

11. The multi-phase oral care system of claim 1, in which
the hydrogel formed by the cross-linking of calcium ions and sodium alginate provides localized adhesion to enamel and sustained release of active agents.

12. The multi-phase oral care system of claim 1, in which:
The cross-linking between calcium ions from Solution A and sodium alginate from Solution B results in a dense hydrogel matrix on the enamel surface;
The cross-linked hydrogel exhibits resistance to dissolution and abrasion during oral use.

13. An enamel surface comprising a cross-linked alginate hydrogel coating layer adhered thereto, wherein:
the hydrogel coating layer comprises ionic cross-links between alginate and calcium ions at the enamel surface; and
the hydrogel coating layer contains one or more active agents selected from antimicrobial agents, breath-freshening agents, or other oral care agents, the active agents being encapsulated within the cross-linked matrix for prolonged release from the adhered hydrogel coating layer.

14. The enamel surface of claim 13, wherein the hydrogel coating layer is formed by applying a calcium-containing solution to the enamel surface to deposit calcium ions, and subsequently applying an alginate-containing solution, wherein interaction between the calcium ions and sodium alginate forms the cross-linked hydrogel coating layer that adheres to the enamel surface and traps active agents selected from antimicrobial agents, breath-freshening agents, or other oral care agents.

15. The enamel surface of claim 13, wherein the hydrogel coating layer provides at least one of:
antimicrobial protection through slow release of at least one antimicrobial agent;
breath freshening through encapsulated breath-freshening agents; or
surface protection against plaque and biofilm formation.

16. The enamel surface of claim 13, wherein the cross-linked hydrogel coating layer is reinforced by an additional application of calcium ions, thereby increasing durability and prolonging active agent retention.

17. The enamel surface of claim 13, wherein the hydrogel coating layer forms a surface-retained coating covering at least a portion of the enamel surface.

18. A removable hydrogel coating on an enamel surface, comprising a cross-linked alginate hydrogel layer adhered to the enamel surface and containing one or more active agents, the hydrogel layer being formed by ionic cross-linking between alginate and calcium ions.

19. The multi-phase oral care system of claim 3, wherein the active agent comprises xylitol at a concentration ranging from 0.5% to 5% (w/v), wherein the xylitol provides antimicrobial protection when retained within the cross-linked hydrogel matrix following A+B or A+B+A application.

20. The multi-phase oral care system of claim 3, wherein the active agent comprises xylitol at a concentration ranging from 5% to 25% (w/v), providing intensive microbial control when retained within the cross-linked hydrogel matrix.
